(19) United States
(12) Patent Application Publication
(10) Pub. No.: US 2022/0313625 A1
(43) Pub. Date: Oct. 6, 2022

(54) PHARMACEUTICAL AGENT, MEDICINAL SOLUTION FOR CLEANING PULMONARY ALVEOLI, AND NEBULIZER

(71) Applicant: The Jikei University, Tokyo (JP)

(72) Inventors: Makiko Ohkido, Tokyo (JP); Naofumi Kimura, Tokyo (JP); Yasushi Mio, Tokyo (JP)

(21) Appl. No.: 17/614,107

(22) PCT Filed: May 21, 2020
(86) PCT No.: PCT/JP2020/020116

§ 371 Date: Nov. 24, 2021

(30) Foreign Application Priority Data
May 24, 2019 (JP) 2019-097756
Jan. 17, 2020 (JP) 2020-006158

(51) Int. Cl. A61K 31/132; A61M 11/02; A61P 11/00; A61K 31/16; A61K 47/24

(52) U.S. Cl. CPC A61K 31/132; A61M 11/02; A61P 11/00; A61K 31/16; A61K 47/24; A61B 1/2676

(57) ABSTRACT

Provided is a pharmaceutical agent for effective prophylaxis and/or treatment of a pathological condition with decreased lung compliance. The pharmaceutical agent for prophylaxis and/or treatment of a pathological condition with decreased lung compliance is characterized by having a polyamine. The polyamine includes at least one of spermine (Spm), spermidine (Spd), and putrescine (Put). The pharmaceutical agent improves lung compliance and ameliorates gas exchange dysfunction, and therefore, can ameliorate pathological conditions such as acute respiratory distress syndrome (ARDS), acute lung injury (ALI), lung diseases caused by dysfunctional endogenous pulmonary alveolar surfactant, multiple organ dysfunction syndrome (MODS), and cardiogenic pulmonary edema.

### TECHNICAL FIELD

The present invention relates to pharmaceutical agents for prophylaxis and/or treatment of a pathological condition with decreased lung compliance, medical solutions for washing pulmonary alveoli having a decrease in lung compliance, and nebulizers.

### BACKGROUND ART

Acute respiratory failure that follows shock or trauma, etc., was reported in 1967 by Ashbaugh et al. Subsequently, the concept of disease called adult respiratory distress syndrome was proposed. The definitions, onset mechanisms, prognosis, etc., of adult respiratory distress syndrome were discussed in 1992 in the American-European Consensus Conference (AECC) organized jointly by the American Thoracic Society and the European Society of Intensive Care Medicine, in which diagnostic criteria were standardized. The ARDS network was established by U.S. National Institutes of Health (NIH), in which the usefulness of low tidal volume artificial ventilation has been proven. In Japan, the “Guidelines for diagnosis and treatment of ALI/ARDS” was prepared in 2005. Acute lung injury (ALI), which is the same pathological condition but called differently, was classified as a mild form of acute respiratory distress syndrome (ARDS) in the Berlin definition in 2012. In Japan, the “Guidelines for diagnosis and treatment of ARDS” was prepared in 2016 based on the Berlin definition.

ARDS is a syndrome including common symptoms associated with causative disease. A hallmark of ARDS is increased permeability pulmonary edema caused by non-specific inflammation occurring in pulmonary alveoli. In these affected pulmonary alveoli, the accumulation of fluid in the alveolar cavities causes impaired oxygenation and decreased lung compliance, leading to respiratory failure. Endogenous pulmonary surfactant in the pulmonary alveoli reduces a force (surface tension) that tends to minimize the surface area of a liquid, and thereby prevents the collapse of the pulmonary alveoli. Dilution and dysfunction of endogenous pulmonary alveolar surfactant are considered to be a critical factor for a decrease, in surface activity that is the surface tension reduction effect, which leads to a decrease in lung compliance.

ARDS is a type of acute respiratory failure having a sudden onset triggered by diseases caused by direct lung injury (pneumonia, accidental swallowing, fat embolism, inhalation injury due to toxic gas or the like, reperfusion pulmonary edema after lung transplantation, drowning, radiation lung injury, pulmonary contusion, etc.) or diseases caused by indirect lung injury (sepsis, severe trauma, thermal burn, shock, massive blood transfusion, drug intoxication, acute pancreatitis, autoimmune disease, etc.), in persons who generally do not have any lung lesion. Patients with ARDS may have severe hypoxemia, increased pulmonary vascular permeability, pulmonary edema, coughing up of frothy bloody sputum, continuous rhonchi in auscultation, etc., and are likely to be affected by sequelae such as encephalopathy, hyaline membrane formation, and pulmonary fibrosis.

Because of compression by heart, dorsal lung region tends to be easy to collapse in ARDS. It is known that lung collapse causes an increase in shunt ratio, which increase in blood flow for which gas exchange does not take place in the lung. Lung collapse also causes the overdistension, thus ventilator-induced lung injury (VILI)). Although it was reported that low tidal volume artificial ventilation in conjunction with positive end-expiratory pressure (PEEP) artificial ventilation significantly reduces the death rate, high PEEP may cause a decrease in cardiac output and an induction of VILI, and the death rate is still high. As a pathological condition similar to ARDS, infantile respiratory distress syndrome (IRDS), which often occurs in preterm infants born before 32 weeks gestational age, is known. Replacement therapy using a bovine lung extract artificial surfactant is effective in treating IRDS, the major cause of which is lack of endogenous surfactant. However, it was reported that replacement therapy using bovine lung extract artificial surfactant or artificial synthetic surfactant is not effective in improving the life prognosis of ARDS, and it has been suspected that there are any other factors involved, in addition to surfactant (NON-PATENT DOCUMENT 5). Furthermore, as pharmacotherapy, a steroid such as methylprednisolone is used in order to reduce inflammation, which is a symptomatic treatment. There is no effective, prophylactic or therapeutic drug for ARDS. Despite the advances in emergency medicine, the present death rate is still as high as 40-50% (NON-PATENT DOCUMENTS 1-4). Therefore, various studies are being extensively conducted to develop a prophylactic or therapeutic drug that is effective in treating ARDS.

### CITATION LIST

### Non-Patent Documents

NON-PATENT DOCUMENT 1: Peter J V, John P, Graham P L, et al. Corticosteroids in the prevention and treatment of acute respiratory distress syndrome (ARDS) in adults: meta-analysis. BMJ. 2008; 336:1006-9.
NON-PATENT DOCUMENT 2: Meduri G U, Golden E, Freire A X, et al. Methylprednisolone infusion in early severe ARDS: results of a randomized controlled trial. Chest. 2007; 131:954-63.
NON-PATENT DOCUMENT 3: Meduri G U, Annane D, Chrousos G P, et al. Activation and regulation of systemic inflammation in ARDS: rationale for prolonged glucocorticoid therapy. Chest. 2009; 136:1631-43.
NON-PATENT DOCUMENT 4: Steinberg K P, Hudson L D, Goodman R B, et al. Efficacy and safety of corticosteroids for persistent acute respiratory distress syndrome. N Engl J Med. 2006; 354:1671-84.
NON-PATENT DOCUMENT 5: Zhang L N, Sun J P, Xue X Y, Wang J X., Exogenous pulmonary surfactant for acute respiratory distress syndrome in adults: A systematic review and meta-analysis. Exp Ther Med. 2013. 5, 237-242.

### SUMMARY OF THE INVENTION

### Technical Problem

With the above-described problems in mind, the present invention has been made. It is an object of the present invention to provide a pharmaceutical agent for prophylaxis and/or treatment of a pathological condition with decreased lung compliance, a medical solution for washing pulmonary alveoli having a decrease in lung compliance, and a nebulizer.

### Solution to the Problem

A pharmaceutical agent according to the present invention is for prophylaxis and/or treatment of a pathological condition with decreased lung compliance, and is characterized by having a polyamine.

A medical solution for washing pulmonary alveoli according to the present invention (note that the medical solution for washing pulmonary alveoli is also referred to as a pulmonary alveolar washing solution) is characterized by having a polyamine.

A nebulizer according to the present invention has an air flow passage that extends from an air introduction opening to a spray opening, and a medical solution reservoir that contains a medical solution and mists the medical solution, wherein air introduced from the air introduction opening is mixed with the medical solution misted in the medical solution reservoir, and the mixed air is discharged from the spray opening, and is characterized in that the medical solution has a polyamine.

### Advantages of the Invention

According to the present invention, a pathological condition with decreased lung compliance can be effectively prevented and/or treated.

### BRIEF DESCRIPTION OF THE DRAWINGS

FIG. 1 is a diagram for describing a structure of a nebulizer according to this embodiment.

FIG. 2 is a diagram for describing a structure of a bronchoscope according to this embodiment.

FIG. 3 is a diagram showing influence of polyamines based on a predetermined polyamine composition, on lung compliance, when the polyamines are administered via the intra-airway route.

FIG. 4 is a diagram showing influence of administration of 0.5 mM of spermine (Spm) on lung compliance.

FIG. 5 is a diagram showing influence of different Spm concentrations on lung compliance.

FIG. 6 is a diagram showing influence of the presence or absence of Spm on lung compliance when a diluted bovine lung extract artificial surfactant is used.

FIG. 7 is a diagram showing the effect of ameliorating gas exchange dysfunction for different Spm concentrations.

FIG. 8 is a diagram showing the effect of ameliorating gas exchange dysfunction using a diluted bovine lung extract artificial surfactant, in the presence or absence of Spm.

FIG. 9 is a photograph showing influence of pulmonary alveolar washing with Spm-containing physiological saline on a lung field image.

FIG. 10 is a diagram showing influence of transairway administration of 5 mM of Spm to an ARDS model animal on lung compliance.

FIG. 11 is a diagram showing curve fitting on a hanging droplet for surface tension measurement.

FIG. 12 is a diagram showing influence of Spm on surface tension in a diluted surfactant environment in which the surface tension reduction effect is weakened.

FIG. 13 is a diagram showing influence of polyamines based on the polyamine composition endogenous to the rat pulmonary alveoli on surface tension.

FIG. 14 is a diagram showing comparison of influence of Spm, and polyamines based on the polyamine composition endogenous to the rat pulmonary alveoli, on surface tension.

FIG. 15 is a diagram showing influence (in vivo effect) of a polyamine having a high in vitro effect (surface tension reduction effect), on lung compliance.

FIG. 16 is a diagram showing influence of spermidine (Spd) and putrescine (Put) on surface tension in a diluted surfactant environment in which the surface tension reduction effect is weakened.

FIG. 17 is a diagram showing influence (60 seconds after formation of a hanging droplet) of polyamines on surface tension in a diluted surfactant environment in which the surface tension reduction effect is weakened.

FIG. 18 is a diagram showing influence of polyamines (an acetylated polyamine, and a polyamine that is not present in the higher animal) on surface tension in a diluted surfactant environment in which the surface tension reduction effect is weakened.

### DESCRIPTION OF EMBODIMENTS

Embodiments of the present invention will be specifically described below with reference to the accompanying drawings. The embodiments are for the purpose of facilitating understanding of the principle of the present invention. The scope of the present invention is not intended to be limited to the embodiments below. Those skilled in the art will make substitutions to the embodiments when necessary without departing the scope of the present invention.

A pharmaceutical agent according to the present invention is for prophylaxis and/or treatment of a pathological condition with decreased lung compliance, and has a polyamine. The present inventors have first found that adding a polyamine(s) to alveoli in which endogenous pulmonary surfactant diluted, in order to adjust to an appropriate concentration of polyamines, allows the lungs to expand and gas exchange to ameliorate. Based on this novel finding, the present invention has been completed.

The pathological condition with decreased lung compliance means, for example, acute respiratory failure in which a chest X-ray photograph taken 12-48 hours after invasion such as trauma or surgery shows pulmonary infiltrative shadow in both of the lungs, and the PaO₂/FiO₂ ratio is at most 300.

The pathological condition with decreased lung compliance is not particularly limited. Examples of the pathological condition with decreased lung compliance include acute respiratory distress syndrome (ARDS), acute lung injury (a mild form of ARDS that used to be called so), and infant respiratory distress syndrome (IRDS). The pathological condition with decreased lung compliance is preferably ARDS in which the PaO₂/FiO₂ ratio is at most 300.

Examples of the pathological condition with decreased lung compliance include lung diseases caused by dysfunctional endogenous pulmonary alveolar surfactant, multiple organ dysfunction syndrome (MODS), and cardiogenic pulmonary edema.

A medical solution for washing pulmonary alveoli according to this embodiment is characterized by having a polyamine.

The polyamine collectively means aliphatic hydrocarbons having two or more primary amino groups. The polyamine is not particularly limited in the present invention. Examples of the polyamine include spermine (Spm), spermidine (Spd), putrescine (Put), and a mixture thereof.

The polyamine can, for example, be acetylputrescine, N1-acetylspermidine, N8-acetylspermidine, N1-acetylspermine, or a mixture thereof.

The polyamine can also, for example, be 1,3-diaminopropane, diaminohexane, cadaverine, agmatine, caldine, homospermidine, aminopropylcadaverine, thermine, thermospermine, canavalmine, aminopentylnorspermidine, N,N-bis(aminopropyl)cadaverine, homospermine, caldopentamine, homocaldopentamine, caldohexamine, homocaldohexamine, or a mixture thereof.

As used herein, the term “prevention” or “prophylaxis” refers to preventing or delaying the onset of a disease or disorder, and includes not only prevention or prophylaxis of occurrence of a disease or disorder, but also prevention or prophylaxis of recurrence of a disease or disorder. As used herein, the term “treatment” or “therapy” refers to eliminating or ameliorating symptoms, and inhibiting the development or progression of symptoms.

The pharmaceutical agent according to this embodiment can be formulated and administered using known techniques. For example, the pharmaceutical agent according to this embodiment can be administered orally or parenterally to humans or mammals in its original liquid form or in an appropriate dosage form. The liquid agent herein includes a product obtained by dissolving a tablet, powder, lyophilized agent, etc., in a solvent (water or physiological saline). In the case of parenteral administration, administration is preferably carried out by inhalation using a nebulizer, artificial ventilator, or inhaler, or by bronchoscope.

The pharmaceutical agent may contain an antiseptic for inhibiting the growth of microorganisms or a buffering agent for maintaining pH in an acceptable range. Examples of the antiseptic include sodium azide, octadecyl dimethyl benzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol. Examples of the buffering agent include phosphoric acid, citric acid, and other organic acids.

The pharmaceutical agent may also contain, for example, an excipient, stabilizer, chelating agent, such as EDTA, salt, or antimicrobial agent. In addition, the pharmaceutical agent can contain antioxidants, such as ascorbic acid and methionine, proteins, such as polypeptides, serum albumin, gelatin, or non-specific immunoglobulins, hydrophilic polymers, such as polyvinyl pyrrolidone, amino acids, such as glycine, glutamine, asparagine, histidine, arginine, and lysine, monosaccharides, disaccharides, and other carbohydrates, such as glucose, mannose, and dextrin, and sugars, such as sucrose, mannitol, trehalose, and sorbitol.

In biological tissue, about 1 mM of polyamines intracellularly synthesized or intestinally absorbed are mainly present in cells. The concentration of extracellular polyamines is about 1/1000- 1/100 of that of intracellular polyamines. It is inferred that polyamines at secretory sites, such as synapses, exist in relatively high concentrations locally. Living organisms have a preserved feedback mechanism for regulating the concentration of intracellular polyamines from becoming too high. Extra polyamines may reduce production of intracellular polyamines. It is, therefore, considered that when a polyamine is administered into pulmonary alveoli for the purpose of prophylaxis and/or treatment, it is preferable that the polyamine concentration at the surface of pulmonary alveoli should not excessively deviate from the intracellular polyamine concentration.

Examples 3 and 5 showed that the presence of 1 mM of Spm at the surface of pulmonary alveoli allowed lung compliance and oxygenation to improved. Therefore, the effective Spm concentration performed at the surface of pulmonary alveoli is considered to be, not limited to, about 1-2 mM.

In administering a polyamine-containing medical solution to a lung affected by ARDS, the efficiency of exchange between exudate retained in pulmonary alveoli and the polyamine-containing medical solution for washing affects the polyamine concentration performed at the surface of pulmonary alveoli. Exchange efficiency is affected by dead volume such as tubes and trachea that do not contribute to the exchange, residual exudate volume, and medical solution volume for washing. Assuming that the polyamine-containing medical solution and the exudate are thoroughly mixed together by a pulmonary alveolar washing operation, a rough calculation indicates that if the exchange efficiency achieved by performing washing once is about 15%, the polyamine concentration produced at the surface of pulmonary alveoli become about 2 mM after washing with a 4-mM polyamine-containing medical solution 5 times consecutively. Thus, it should be considered and determined that the concentration of polyamine-containing medical solution administered will be diluted by exudate. Example 7 shows an effect obtained when washing with a medical solution containing 5 mM of Spm was carried out 5 times consecutively on ARDS rats produced by pulmonary alveolar washing with physiological saline. Although not particularly limited, in the case where the polyamine contained in the medical solution is Spm, the selected medical solution concentration is about 1-50 mM, 1-10 mM, or 1-5 mM for intralesional use, or about 1-10 mM, preferably about 1-5 mM, for use in a wide region including a lesion.

The pharmaceutical agent according to the present invention can be incorporated into liposomes for drug delivery. The liposome contains, for example, a phospholipid, such as a phosphatidylserine (PS) or phosphatidylcholine (PC), as a membrane component. The diameter of the liposome can be suitably adjusted, taking into account the success rate of delivery to target tissue, stability, etc. The liposome is, for example, a monolayer liposome having a diameter of 150-350 nm.

The pharmaceutical agent according to the present invention has a polyamine, and can further contain a phospholipid component or a phospholipid. The major component of endogenous pulmonary surfactant is phospholipids. A commercially available bovine lung extract artificial surfactant contains 84% of phospholipids.

The phospholipid component and phospholipid are not particularly limited. Examples of the phospholipid component and phospholipid include dipalmitoyl-phosphatidylcholine (DPPC), phosphatidylcholines (PC), phosphatidylglycerols (PG), distearoylphosphatidylcholine (DSPC), distearoylphosphatidylglycerol (DSPG), dimyristoyl-phosphatidylethanolamine (DMPE), dipalmitoylphosphatidylethanolamine (DPPE), dioleylphosphatidylethanolamine (DOPE), distearoylphosphatidylethanolamine (DSPE), diarachidoylphosphatidylethanolamine (DAPE) or dilinoleylphosphatidylethanolamine (DLPE), dilauroyl-phosphatidylcholine (DLPC), dimyristoyl-phosphatidylcholine (DMPC), diarachidoyl-phosphatidylcholine (DAPC), dioleyl-phosphatidylcholine (DOPC), dimyristoylphosphatidylserine (DMPS), diarachidoylphosphatidylserine (DAPS), dipalmitoylphosphatidylserine (DPPS), distearoylphosphatidylserine (DSPS), dioleylphosphatidylserine (DOPS), dipalmitoylphosphatidic acid (DPPA), dimyristoylphosphatidic acid (DMPA), distearoylphosphatidic acid (DSPA), diarachidoylphosphatidic acid (DAPA), dimyristoylphosphatidylglycerol (DMPG), dipalmitoylphosphatidylglycerol (DPPG), dioleyl-phosphatidylglycerol (DOPG), dilauroylphosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), dioleylphosphatidylinositol (DOPI), and a mixture thereof.

It has been reported that the pulmonary surfactant replacement therapy is not effective in treating, for example, ARDS. However, it is effective to use the pulmonary surfactant replacement therapy in conjunction with the pharmaceutical agent having a polyamine according to the present invention for treatment of ARDS.

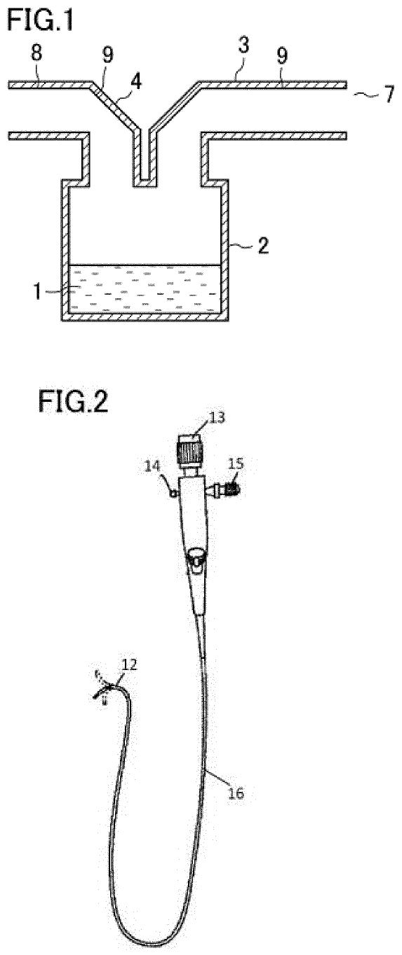

As shown in FIG. 1, the nebulizer according to this embodiment has an air flow passage 9 that extends from an air introduction opening 7 to a spray opening 8, and a medical solution reservoir 2 that contains a medical solution 1 and mists the medical solution 1. Air introduced from the air introduction opening 7 is mixed with the medical solution 1 misted in the medical solution reservoir 2, and the mixed air is discharged from the spray opening 8. The nebulizer is characterized in that the medical solution 1 has a polyamine.

As shown in FIG. 1, the nebulizer has the medical solution reservoir 2 for containing the medical solution 1 to be misted and sprayed, and two tubes 3 and 4 that are provided at an upper portion of the medical solution reservoir 2 and are in communication with the inside of the medical solution reservoir 2. The medical solution reservoir 2 is provided with an ultrasonic vibrator for misting the medical solution 1 contained therein, for example, at a lower portion of the medical solution reservoir 2. The misting of the medical solution 1 is accelerated by ultrasonic vibration produced by the ultrasonic vibrator. One of the two tubes 3 and 4 provided at the upper portion of the medical solution reservoir 2 is an air introduction tube 3 one end opening of which serves as the air introduction opening 7. The other tube is an air discharge tube 4 one end opening of which serves as the spray opening 8. The air introduction tube 3 and the air discharge tube 4 are bent into an L shape, extending in opposite directions away from each other. The air flow passage 9 is defined as the passage extending from the air introduction opening 7 to the spray opening 8 through the air introduction tube 3, the medical solution reservoir 2, and the air discharge tube 4. Administration of a medical solution using a nebulizer allows the medical solution to spread throughout the lungs, and therefore, is suitable for treatment of mild forms of pathological conditions. Therefore, when administration is carried out using a nebulizer, the concentration of a medical solution can be reduced. Although not particularly limited, the polyamine concentration can, for example, be 1-5 mM.

As shown in FIG. 2, a bronchoscope according to this embodiment has a tip portion 12 that bends flexibly, and an ocular portion 13 at an base end thereof. An operation portion 14 is used to bend the tip portion 12. The bronchoscope includes a tube-shaped insertion tube portion 16 that is inserted into the trachea and then a bronchus, through the mouth or nose and then the throat, or through a tracheal cannula. The insertion tube portion 16, which is flexible and in the shape of a tube, is provided with a spray hole penetrating from a rear end to a front end of the tube. The medical solution is transferred through the spray hole. The medical solution is characterized by having a polyamine. A spray opening that is the front end of the spray hole is provided at the tip portion 12. The medical solution is fed in the form of a mist from the spray opening to pulmonary alveoli. A feed opening 15 for feeding the medical solution into the spray hole of the insertion tube portion 16 is provided at the base end.

Administration of a medical solution using a bronchoscope is locally available to a lesional portion, each lobe, or each lung, which is suitable for treatment of pathological conditions with a severe symptom. Therefore, in the case of administration using a bronchoscope, a high-concentration medical solution can be used. Although not particularly limited, the polyamine concentration can, for example, be 2-100 mM. If it is difficult to administer using a bronchoscope in severe cases, it is possible to do using a nebulizer as described above.

### EXAMPLES

### (1) Example 1

In Example 1, the effect of improving lung compliance by polyamines administered to pulmonary alveoli was studied based on the a polyamine composition ratio (Put:Spd:Spm=0.1:3:2) in pulmonary alveoli of a euthanized mouse.

A tracheal cannula was inserted into a rat under general anesthesia. When the respiratory and circulatory dynamics of the rat were stable under artificial ventilation, an injection/suction operation using 20 ml/kg of a pulmonary alveolar washing solution (physiological saline) through the tracheal cannula was conducted 3 times consecutively within 2 minutes. After the washing solution remaining in the trachea was suctioned out, artificial ventilation was immediately resumed. Thus, an ARDS model whose pulmonary alveoli were washed with physiological saline was produced. The pulmonary alveolar washing operation means production of a respiratory distress syndrome (ARDS) model having decreased lung compliance.

A tracheal cannula was inserted into a rat under general anesthesia. When the respiratory and circulatory dynamics of the rat were stable under artificial ventilation, 20 ml/kg of a pulmonary alveolar washing solution (physiological saline containing 0.51 mM of polyamines (composition ratio is Put:Spd:Spm=0.1:3:2) was injected/suctioned consecutively 3 times within 2 minutes through the tracheal cannula. After the washing solution remaining in the trachea was suctioned out, artificial ventilation was immediately resumed. Thus, an ARDS model whose pulmonary alveoli were washed with the polyamines was produced. The pulmonary alveolar washing with a polyamine-containing physiological saline means production of an ARDS model and administration of polyamines to pulmonary alveoli, which are simultaneously carried out.

The above ventilation was conducted by inverse ratio ventilation without using PEEP. The ventilation was conducted using ambient air (inhaled oxygen concentration FiO₂: 0.21) without oxygen administration. The intra-airway pressure and the partial pressure of oxygen in the arterial blood (PaO₂) were measured over time. Influence of the polyamines was analyzed, where dynamic lung compliance (=a tidal volume/a change in intra-airway pressure) (expression 1) was used as a measure of the distensibility of the lung, and PaO₂/FiO₂ (P/F ratio)<300 was used as a criterion for gas exchange dysfunction.

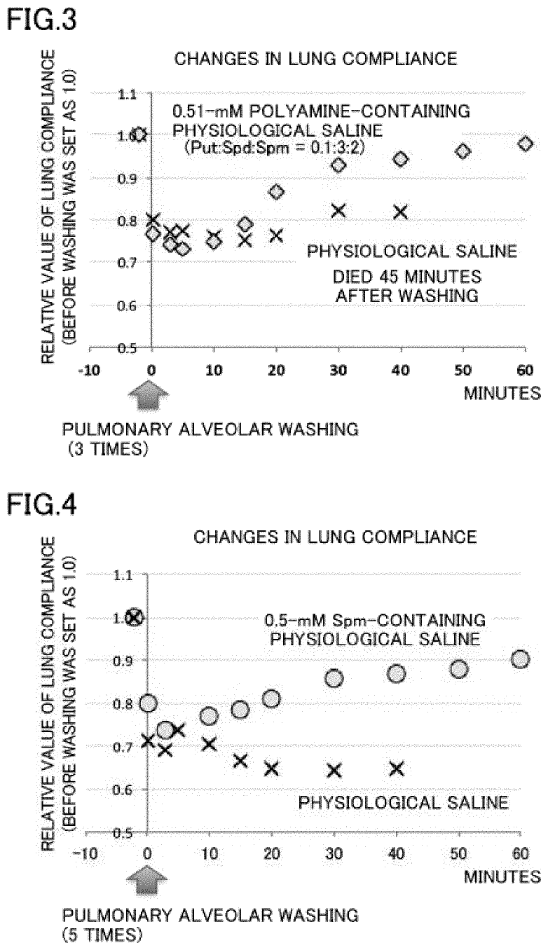

The result is shown in FIG. 3. As shown in FIG. 3, lung compliance decreased immediately after washing due to the pulmonary alveolar washing operation (closed gray diamonds and cross in FIG. 3). In the case of pulmonary alveolar washing with physiological saline containing 0.51 mM of polyamines (Put:Spd:Spm=0.1:3:2), lung compliance subsequently recovered (closed gray diamonds in FIG. 3).

### (2) Example 2

In Example 2, the effect of improving lung compliance by a polyamine (0.5 mM of Spm) administered to pulmonary alveoli was studied.

A tracheal cannula was inserted into a rat under general anesthesia. When the respiratory and circulatory dynamics of the rat were stable under artificial ventilation, 20 ml/kg of a pulmonary alveolar washing solution (physiological saline) was injected/suctioned consecutively 5 times within 2 minutes through the tracheal cannula. After the washing solution remaining in the trachea was suctioned out, artificial ventilation was immediately resumed. Thus, an ARDS model whose pulmonary alveoli were washed with physiological saline was produced.

A tracheal cannula was inserted into a rat under general anesthesia. When the respiratory and circulatory dynamics of the rat were stable under artificial ventilation, 20 ml/kg of a pulmonary alveolar washing solution (physiological saline containing 0.5 mM of Spm) was injected/suctioned consecutively 5 times within 2 minutes through the tracheal cannula. After the washing solution remaining in the trachea was suctioned out, artificial ventilation was immediately resumed. Thus, an ARDS model whose pulmonary alveoli were washed with 0.5 mM of Spm was produced.

The above ventilation was conducted by inverse ratio ventilation without using PEEP. The ventilation was conducted using ambient air (inhaled oxygen concentration FiO₂: 0.21) without oxygen administration. The intra-airway pressure and the partial pressure of oxygen in the arterial blood (PaO₂) were measured over time. Influence of the polyamine was analyzed, where dynamic lung compliance was used as a measure of the distensibility of the lung, and the P/F ratio <300 was used as a criterion for gas exchange dysfunction.

Influence of the polyamine was analyzed in a manner similar to that of Example 1.

The result is shown in FIG. 4. As shown in FIG. 4, lung compliance decreased immediately after washing due to the pulmonary alveolar washing operation (closed gray circles and cross in FIG. 4). In the case of pulmonary alveolar washing with physiological saline containing 0.5 mM of Spm, lung compliance subsequently recovered (closed gray circles in FIG. 4).

### (3) Example 3

In Example 3, the effect of improving lung compliance, depending on the concentration of Spm administered to pulmonary alveoli, was compared and studied.

A tracheal cannula was inserted into a rat under general anesthesia. When the respiratory and circulatory dynamics of the rat were stable under artificial ventilation, 20 ml/kg of a pulmonary alveolar washing solution (physiological saline) was injected/suctioned consecutively 5 times within 2 minutes through the tracheal cannula. After the washing solution remaining in the trachea was suctioned out, artificial ventilation was immediately resumed. Thus, an ARDS model whose pulmonary alveoli were washed with physiological saline was produced.

A tracheal cannula was inserted into a rat under general anesthesia. When the respiratory and circulatory dynamics of the rat were stable under artificial ventilation, 20 ml/kg of a pulmonary alveolar washing solution (physiological saline containing 1 mM of Spm) was injected/suctioned consecutively 5 times within 2 minutes through the tracheal cannula. After the washing solution remaining in the trachea was suctioned out, artificial ventilation was immediately resumed. Thus, an ARDS model whose pulmonary alveoli were washed with 1 mM of Spm was produced.

A tracheal cannula was inserted into a rat under general anesthesia. When the respiratory and circulatory dynamics of the rat were stable under artificial ventilation, 20 ml/kg of a pulmonary alveolar washing solution (physiological saline containing 0.5 mM of Spm) was injected/suctioned consecutively 5 times within 2 minutes through the tracheal cannula. After the washing solution remaining in the trachea was suctioned out, artificial ventilation was immediately resumed. Thus, an ARDS model whose pulmonary alveoli were washed with 0.5 mM of Spm was produced.

The above ventilation was conducted by inverse ratio ventilation without using PEEP. The ventilation was conducted using ambient air (inhaled oxygen concentration FiO₂: 0.21) without oxygen administration. The intra-airway pressure and the partial pressure of oxygen in the arterial blood (PaO₂) were measured over time. Influence of the polyamine was analyzed, where dynamic lung compliance was used as a measure of the distensibility of the lung, and the P/F ratio <300 was used as a criterion for gas exchange dysfunction.

Influence of the polyamine was analyzed in a manner similar to that of Example 1.

The result is shown in FIG. 5. As shown in FIG. 5, lung compliance decreased immediately after washing due to pulmonary alveolar washing (open circles, closed gray circles, and cross in FIG. 5). In all the cases of pulmonary alveolar washing with Spm-containing physiological saline, lung compliance subsequently recovered (open circles and closed gray circles in FIG. 5). The effect of improving lung compliance was higher for 1 mM of Spm (open circles in FIG. 5) than for 0.5 mM of Spm (closed gray circles in FIG. 5) until 60 minutes have passed since washing. When lung compliance before washing was set as 1.0, the time it took to recover to 0.9 after washing with 0.5 mM of Spm (closed gray circles in FIG. 5) was an average of 60 minutes, but that with 1 mM of Spm (open circles in FIG. 5) was as short as an average of 15 minutes. There were some samples in which after 30 minutes has passed since washing, lung compliance was further improved, exceeding 1.0, which was lung compliance before washing.

### (4) Example 4

In Example 4, an additional experiment was conducted to demonstrate whether diluted surfactant does not have the effect of improving lung compliance, and that the presence of 1 mM of Spm does.

A tracheal cannula was inserted into a rat under general anesthesia. When the respiratory and circulatory dynamics of the rat were stable under artificial ventilation, 20 ml/kg of a pulmonary alveolar washing solution (0.3 mg/ml of a bovine lung extract artificial surfactant; the bovine lung extract artificial surfactant was formulated by dissolving Surfacten (trade name), manufactured by Mitsubishi Tanabe Pharma Corporation, in physiological saline to a concentration of 0.3 mg/ml, which is 1/100 of the concentration that is normally for administration to a preterm infant) was injected/suctioned consecutively 5 times within 2 minutes through the tracheal cannula. After the washing solution remaining in the trachea was suctioned out, artificial ventilation was immediately resumed. Thus, an ARDS model whose pulmonary alveoli were washed with the diluted bovine lung extract artificial surfactant was produced. Note that it was confirmed that no polyamines are detected in the bovine lung extract artificial surfactant.

A tracheal cannula was inserted into a rat under general anesthesia. When the respiratory and circulatory dynamics of the rat were stable under artificial ventilation, 20 ml/kg of a pulmonary alveolar washing solution (0.3 mg/ml of a bovine lung extract artificial surfactant containing 1 mM of Spm; the bovine lung extract artificial surfactant was Surfacten (trade name), manufactured by Mitsubishi Tanabe Pharma Corporation) was injected/suctioned consecutively 5 times within 2 minutes through the tracheal cannula. After the washing solution remaining in the trachea was suctioned out, artificial ventilation was immediately resumed. Thus, an ARDS model whose pulmonary alveoli were washed with the diluted bovine lung extract artificial surfactant containing 1 mM of Spm was produced.

The above ventilation was conducted by inverse ratio ventilation without using PEEP. The ventilation was conducted using ambient air (inhaled oxygen concentration FiO₂: 0.21) without oxygen administration. The intra-airway pressure and the partial pressure of oxygen in the arterial blood (PaO₂) were measured over time. Influence of the polyamine was analyzed, where dynamic lung compliance was used as a measure of the distensibility of the lung, and the P/F ratio <300 was used as a criterion for gas exchange dysfunction.

Influence of the polyamine was analyzed in a manner similar to that of Example 1.

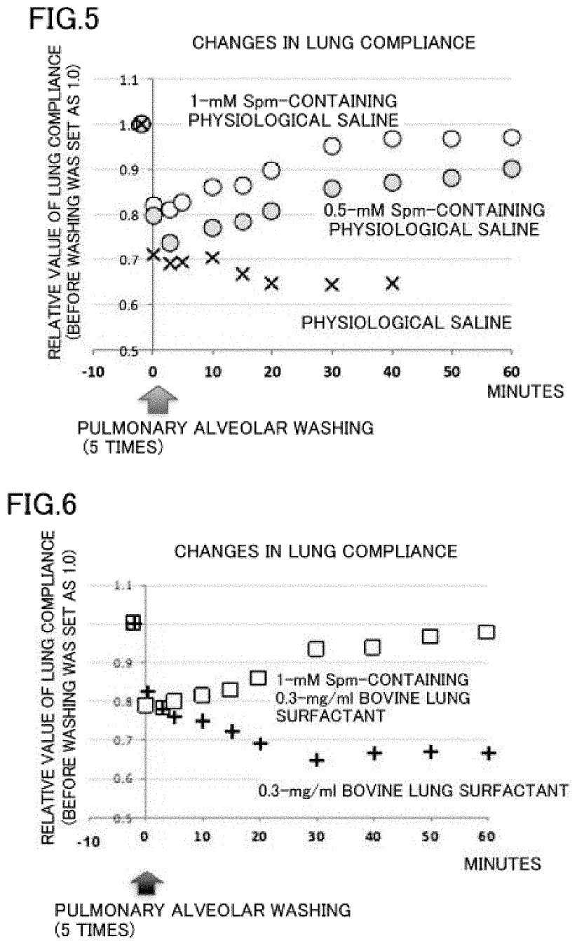

The result is shown in FIG. 6. In FIG. 6, bovine lung extract artificial surfactant is represented by bovine lung surfactant. As shown in FIG. 6, lung compliance decreased immediately after washing due to pulmonary alveolar washing (open squares and plus in FIG. 6). In the case of pulmonary alveolar washing with the 0.3 mg/ml of a bovine lung surfactant containing 1 mM of Spm, lung compliance subsequently recovered (open squares in FIG. 6). In the case of pulmonary washing with the diluted bovine lung extract artificial surfactant alone, the recovery of lung compliance was not observed even 60 minutes after the washing. When lung compliance before washing was set as 1.0, the time it took to recover to 0.9 after washing with the 0.3 mg/ml of a bovine lung surfactant containing 1 mM of Spm was an average of 20 minutes (open squares in FIG. 6). Examples 3 and 4 demonstrate that the presence of Spm in the washing solution improves lung compliance no matter whether or not a bovine lung surfactant is present.

### (5) Example 5

In Example 5, the effect of improving gas exchange by administering Spm to pulmonary alveoli was studied.

An ARDS model whose pulmonary alveoli were washed with physiological saline, an ARDS model whose pulmonary alveoli were washed with 1 mM of Spm, and an ARDS model whose pulmonary alveoli were washed with 0.5 mM of Spm, were produced in a manner similar to that of Example 3.

An ARDS model whose pulmonary alveoli were washed with a 0.3 mg/ml bovine lung surfactant, and an ARDS model whose pulmonary alveoli were washed with a 0.3 mg/ml bovine lung surfactant containing 1 mM of Spm, were produced in a manner similar to that of Example 4.

Ventilation was conducted by inverse ratio ventilation without using PEEP. The ventilation was conducted using ambient air (inhaled oxygen concentration FiO₂: 0.21) without oxygen administration. The intra-airway pressure and the partial pressure of oxygen in the arterial blood (PaO₂) were measured over time. Influence of the polyamine was analyzed, where dynamic lung compliance was used as a measure of the distensibility of the lung, and the P/F ratio <300 was used as a criterion for gas exchange dysfunction.

Arterial blood sampling was conducted before pulmonary alveolar washing, 20 minutes after the washing, and 60 minutes after the washing, and the partial pressure of oxygen in the arterial blood (PaO₂) was measured using a blood gas analyzer. Influence of Spm administered to pulmonary alveoli on the rate of occurrence of gas exchange dysfunction was analyzed, where the P/F ratio <300 was used as a criterion for gas exchange dysfunction. A decrease in percutaneous arterial oxygen saturation (SpO₂), which has a correlation with PaO₂, was observed in an oxygen dissociation curve. Cases (death) that death occurred before the measurement times, i.e., arterial blood sampling was not conducted, were counted as the case of occurrence of gas exchange dysfunction.

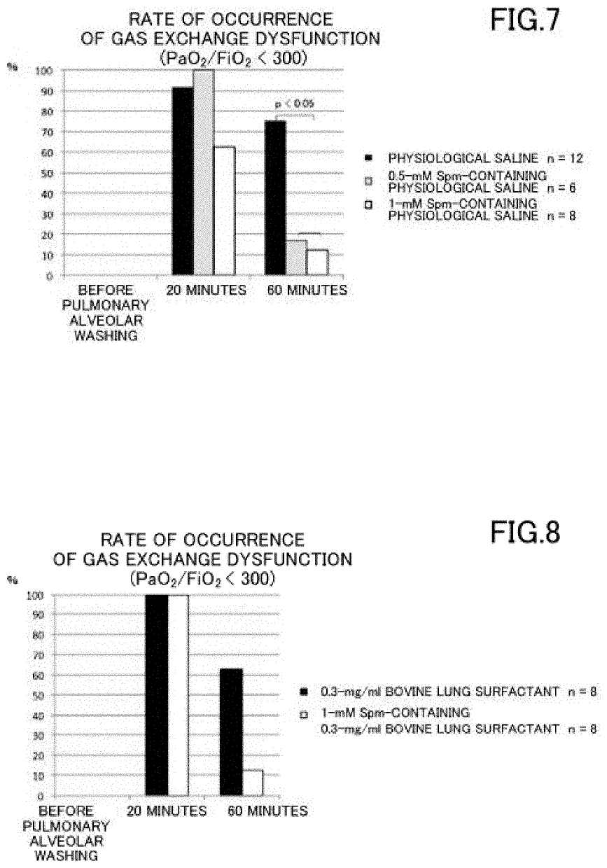

The result is shown in FIG. 7. As shown in FIG. 7, in the case of pulmonary alveolar washing with the Spm-containing physiological saline, the gas exchange dysfunction occurrence rate 1 hour after the washing significantly decreased (gray columns and open columns (60 minutes) in FIG. 7), compared to washing with physiological saline (closed columns in FIG. 7). In the case where 1 mM of Spm was contained (open columns in FIG. 7), the gas exchange dysfunction occurrence rate 20 minutes after the washing was lower than the others (20 minutes in FIG. 7).

The result is shown in FIG. 8. As shown in FIG. 8, in the case of pulmonary alveolar washing with the 0.3 mg/ml bovine lung surfactant containing 1 mM of Spm (open columns in FIG. 8), the gas exchange dysfunction occurrence rate 1 hour after the washing decreased, compared to washing with the 0.3 mg/ml bovine lung surfactant alone (closed columns in FIG. 8).

Thus, it was demonstrated that administration of Spm to pulmonary alveoli has the effect of improving gas exchange.

### (6) Example 6

In Example 6, influence of pulmonary alveolar washing with Spm-containing physiological saline on a lung field image was studied. Plain X-ray imaging and X-ray CT imaging were performed, with an air pressure (0 and 20 cm H₂O) exerted through a tracheal cannula, on rats that were euthanized after pulmonary alveolar washing or rats that were subjected to pulmonary alveolar washing immediately after euthanization.

A rat a was a control that was not subjected to pulmonary alveolar washing. Specifically, a tracheal cannula was inserted into a rat under general anesthesia, and the rat was euthanized without pulmonary alveolar washing.

A rat b was subjected to pulmonary alveolar washing with physiological saline containing 1 mM of Spm. Specifically, a tracheal cannula was inserted into a rat under general anesthesia, and after the rat was euthanized, 20 ml/kg of a pulmonary alveolar washing solution (physiological saline containing 1 mM of Spm) was injected/suctioned consecutively 5 times within 2 minutes through the tracheal cannula, and the washing solution remaining in the trachea was suctioned out.

A rat c was subjected to pulmonary alveolar washing with physiological saline. Specifically, a tracheal cannula was inserted into a rat under general anesthesia. When the respiratory and circulatory dynamics of the rat were stable under artificial ventilation, 20 ml/kg of a pulmonary alveolar washing solution (physiological saline) was injected/suctioned consecutively 5 times within 2 minutes through the tracheal cannula. After the washing solution remaining in the trachea was suctioned out, artificial ventilation was immediately resumed, and 30 minutes after that, the rat was euthanized.

A rat d was subjected to pulmonary alveolar washing with physiological saline, and then pulmonary alveolar washing with physiological saline containing 5 mM of Spm. Specifically, a tracheal cannula was inserted into a rat under general anesthesia, and after the rat was euthanized, 20 ml/kg of a pulmonary alveolar washing solution (physiological saline) was injected/suctioned consecutively 5 times within 2 minutes through the tracheal cannula, and the washing solution remaining in the trachea was suctioned out. Following this, an injection/suction operation using 20 ml/kg of a pulmonary alveolar washing solution (physiological saline containing 5 mM of Spm) through the tracheal cannula was conducted 5 times consecutively within 2 minutes, and the washing solution remaining in the trachea was suctioned out.

The imaging device was an experimental animal X-ray CT device, Latheta LCT-200. The imaging conditions were as follows: the voxel size was 120 pin; the angle of rotation was 360°; the rotational speed was normal; and the X-ray tube voltage was low.

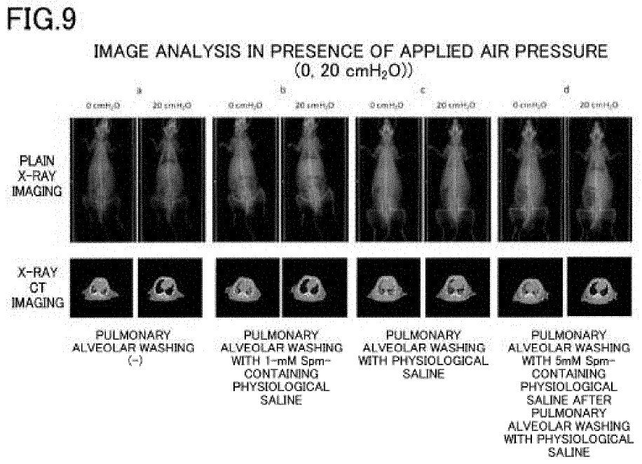

The result was shown in FIG. 9. As shown in FIG. 9, due to pulmonary alveolar washing, the density of the lung field increased (lung field density: a<<b and c<d in CT images, where 0 cm H₂O) in CT images in the absence of applied pressure. In the X-ray images captured in the presence of an applied pressure of 20 cm H₂O, of the rat c whose pulmonary alveoli were washed with physiological saline, the cardiac shadow was unclear, and the transmittance of the lung field decreased. In the CT images thereof, infiltrative shadows, which indicate a high absorption value, non-uniformly appeared in the lung field, and the volume of air in the lungs decreased. In the X-ray images captured in the presence of an applied pressure of 20 cm H₂O, of the rat d whose pulmonary alveoli were washed with physiological saline and then with physiological saline containing 5 mM of Spm, the transmittance of the lung field was somewhat poor compared to the control, and the cardiac shadow became clear. In the CT images thereof, no wide range of infiltrative shadow was observed unlike the rat c, and air was relatively well throughout the lungs. The rat d was subjected to pulmonary alveolar washing twice. It is inferred that although the dilution ratio of endogenous surfactant was higher in the rat d than in the rats b and c, the volume of air in the lung field was recovered by Spm administered to pulmonary alveoli in the second pulmonary alveolar washing. Thus, it is concluded that administration of Spm to pulmonary alveoli has the effect on aeration: recruitment, of a collapsed lung that causes VALI or an increase in shunt ratio.

### (7) Example 7

In Example 7, influence of transairway administration of 5 mM of Spm to an ARDS model animal on lung compliance was studied.

It was demonstrated in Example 6 that the transairway administration of 5 mM of Spm by pulmonary alveolar washing with physiological saline containing 5 mM of Spm made the air in the lung field in ARDS model animal increase in a CT image captured in the presence of an applied pressure of 20 cm H₂O (FIG. 9d). Given that dynamic lung compliance equals a tidal volume divided by a change in intra-airway pressure (expression 1), it is inferred that the above phenomenon is caused by an increase in tidal volume of the lungs: the volume of air in the lungs, in the presence of an intra-airway pressure of 20 cm H₂O. If this is the case, the lung compliance must have increased after pulmonary alveolar washing with physiological saline containing 5 mM of Spm. This was studied in Example 7. Specifically, influence of transairway administration of 5 mM of Spm to an ARDS model animal on lung compliance was analyzed.

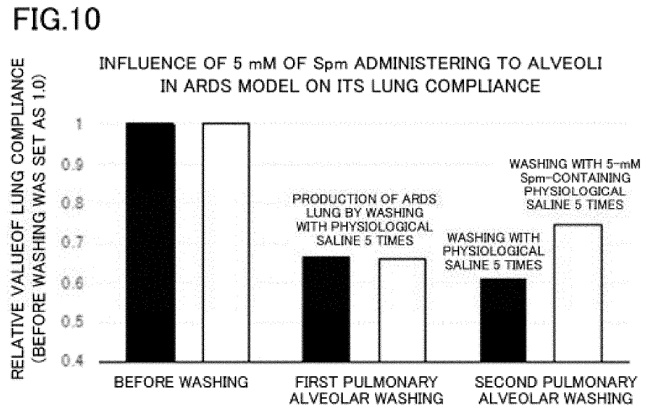

A tracheal cannula was inserted into a rat under general anesthesia, and then euthanized. Immediately after that, artificial ventilation was conducted for 30 seconds, and lung compliance before washing that is calculated based on intra-airway pressure was set as 1.0 (“before washing” on the left slide of FIG. 10). Following this, a lung affected by ARDS was produced. Specifically, an injection/suction operation was conducted 5 times consecutively within 2 minutes using 20 ml/kg of physiological saline through the tracheal cannula. After the washing solution remaining in the tracheal cannula was suctioned out, artificial ventilation was conducted for 30 seconds, and the lung compliance of the lung affected by ARDS was calculated and compared with that of the normal lung (“first pulmonary alveolar washing” at the center of FIG. 10). Following this, the lung affected by ARDS was subjected to transairway administration of 5 mM of Spm, and any therapeutic effect was studied. Specifically, an injection/suction operation was conducted 5 times consecutively within 2 minutes using 20 ml/kg of physiological saline containing 5 mM of Spm through the tracheal cannula. After the washing solution remaining in the tracheal cannula was suctioned out, artificial ventilation was conducted for 30 seconds, and the lung compliance calculated based on intra-airway pressure (“second pulmonary alveolar washing” (open column) on the right side of FIG. 10) was compared with that of the lung affected by ARDS. Lung compliance obtained when a lung affected by ARDS was subjected to transairway administration of physiological saline was a control (“second pulmonary alveolar washing” (closed column) on the right side of FIG. 10).

The result is shown in FIG. 10. The transairway administration of 5 mM of Spm to a lung affected by ARDS and having decreased lung compliance improved the lung compliance (open column on the right side of FIG. 10). Meanwhile, the transairway administration of physiological saline to a lung affected by ARDS further decreased the lung compliance (closed column on the right side of FIG. 10).

It was demonstrated in Examples 6 and 7 that the technique of administration by pulmonary alveolar washing is effective as a form of administration of a polyamine to a lung affected by ARDS. If a bronchoscope is used, bronchoalveolar lavage (BAL) allows localized administration to a lesion. BAL also allows removal of inflammation cytokines and exudates before polyamine administration. Example 7 shows the possibility that the lung compliance of a patient decreases on BAL test that is conducted by alveolar washing with physiological saline. Examples 6 and 7 indicate that at the last bronchial washing operation, BAL using a physiological saline containing polyamine can prevent pulmonary collapse, one of the complications.

### (8) Example 8

In Example 8, influence of a polyamine on surface tension in a diluted surfactant environment in which the surface tension reduction effect is weakened, was studied.

It is considered that in ARDS, the main cause for the collapse of pulmonary alveoli and decreased lung compliance is that the “surface tension reduction effect” exhibited by endogenous pulmonary surfactant is weakened by exudate accumulated in pulmonary alveolar cavities. In Examples 6 and 7, the transairway administration of 5 mM of Spm to a lung affected by ARDS increased the volume of air in the lung and improved compliance. Therefore, it was inferred that 5 mM of Spm has the effect of recovering the “surface tension reduction effect” that was weakened due to dilution of endogenous surfactant. This inference was studied in Example 8 in an in vitro experimental system using a bovine lung extract artificial surfactant. In other words, it was studied whether a polyamine reduces the “increased surface tension” due to dilution of a bovine lung surfactant.

Surface tension was measured by the Young-Laplace method (curve fitting) using a contact angle meter (B100, manufactured by Asumi Giken, Limited), on a droplet formed at the tip of a 20-gauge straight needle.

Tensiometer: contact angle meter (B100, manufactured by Asumi Giken, Limited)

Surface tension measurement method: Young-Laplace method (curve fitting)

Hanging droplet production method: a maximum size of droplet not to drop was produced at the tip of a 20-gauge straight needle (an autodispenser was used)

Imaging of hanging droplet: imaging was performed using a CCD camera

Bovine lung surfactant: a commercially available bovine lung extract artificial surfactant (Surfacten (trade name), manufactured by Mitsubishi Tanabe Pharma Corporation) was adjusted to a concentration of 30 mg/ml using physiological saline according to the package insert. The concentration of 30 mg/ml is one for treatment of a preterm infant who has pulmonary alveoli collapsed due to lack of pulmonary surfactant. In the drawings, the formulated surfactant solution is indicated by “30 mg/ml surf.”

10-fold diluted bovine lung surfactant: a commercially available bovine lung extract artificial surfactant (Surfacten (trade name), manufactured by Mitsubishi Tanabe Pharma Corporation) was adjusted to a concentration of 3 mg/ml using physiological saline. In the drawings, the formulated surfactant solution is indicated by “3 mg/ml surf.”

100-fold diluted bovine lung surfactant: a commercially available bovine lung extract artificial surfactant (Surfacten (trade name), manufactured by Mitsubishi Tanabe Pharma Corporation) was adjusted to a concentration of 0.3 mg/ml using physiological saline. Pulmonary surfactant was diluted with physiological saline, i.e., the resultant solution mimics the state of surfactant in a lung affected by ARDS. In the drawings, the formulated surfactant solution is indicated by “100-fold diluted bovine lung surfactant,” “0.3-mg/ml bovine lung surfactant,” “0.3 mg/ml surf,” or “ 1/100 Surf.”

1000-fold diluted bovine lung surfactant: a commercially available bovine lung extract artificial surfactant (Surfacten (trade name), manufactured by Mitsubishi Tanabe Pharma Corporation) was adjusted to a concentration of 0.03 mg/ml using physiological saline. In the drawings, the formulated surfactant solution is indicated by “0.03 mg/ml surf.”

It was verified that no polyamines were detected in the bovine lung surfactant.

Polyamine-containing 100-fold diluted bovine lung surfactant:

Spermine, which is a representative example of a major polyamine present in the higher animals (claim 3), N1-acetylspermidine, which is a representative example of an acetylated polyamine (claim 4), or diaminohexane, which is a representative example of a polyamine which is not present in the higher animals (claim 5), was added to a diluted pulmonary Surfacten solution, and the presence or absence of the surface tension reduction effect was analyzed. The solution was formulated using a commercially available polyamine chloride.

A spermine-containing 100-fold diluted bovine lung surfactant is indicated, in the drawings and specification, by “Spm-containing 0.3 mg/ml bovine lung surfactant,” “Spm-containing 1/100 surf,” or “Spm-containing 0.3 mg/ml surf.”

A spermidine-containing 100-fold diluted bovine lung surfactant is indicated, in the drawings and specification, by “Spd-containing 0.3 mg/ml bovine lung surfactant,” Spd-containing 1/100 surf,” or “Spd-containing 0.3 mg/ml surf.”

A putrescine-containing 100-fold diluted bovine lung surfactant is indicated, in the drawings and specification, by “Put-containing 0.3 mg/ml bovine lung surfactant,” “Put-containing 1/100 surf,” or “Put-containing 0.3 mg/ml surf.”

An N1-acetylspermidine-containing 100-fold diluted bovine lung surfactant is indicated, in the drawings and specification, by “N1-AcSpd-containing 0.3 mg/ml bovine lung surfactant,” “AcSpd-containing 1/100 surf,” or “AcSpd-containing 0.3 mg/ml surf.”

A 1-mM diaminohexane-containing 100-fold diluted bovine lung surfactant is indicated, in the drawings and specification, by “diaminohexane-containing 0.3 mg/ml bovine lung surfactant,” “diaminohexane-containing 1/100 surf,” or “diaminohexane-containing 0.3 mg/ml surf.”

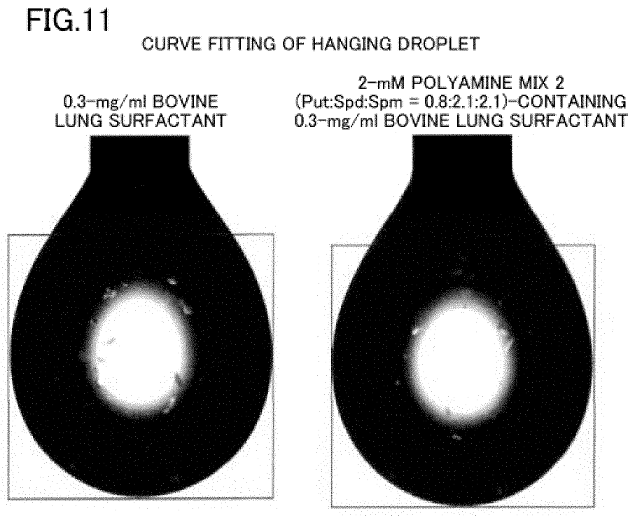

The results are shown in FIGS. 11, 12, 16, and 17. FIG. 11 shows images that droplets derived from a 0.3-mg/ml bovine lung surfactant and a 2-mM polyamine mix 2-containing 0.3-mg/ml bovine lung surfactant (described below in Example 9), were captured after the lapse of a predetermined period of time. The droplet containing 2 mM of the polyamine mix 2 extended further in the gravitational direction. The surface tension of a droplet was calculated by performing curve fitting on the shape of the droplet.

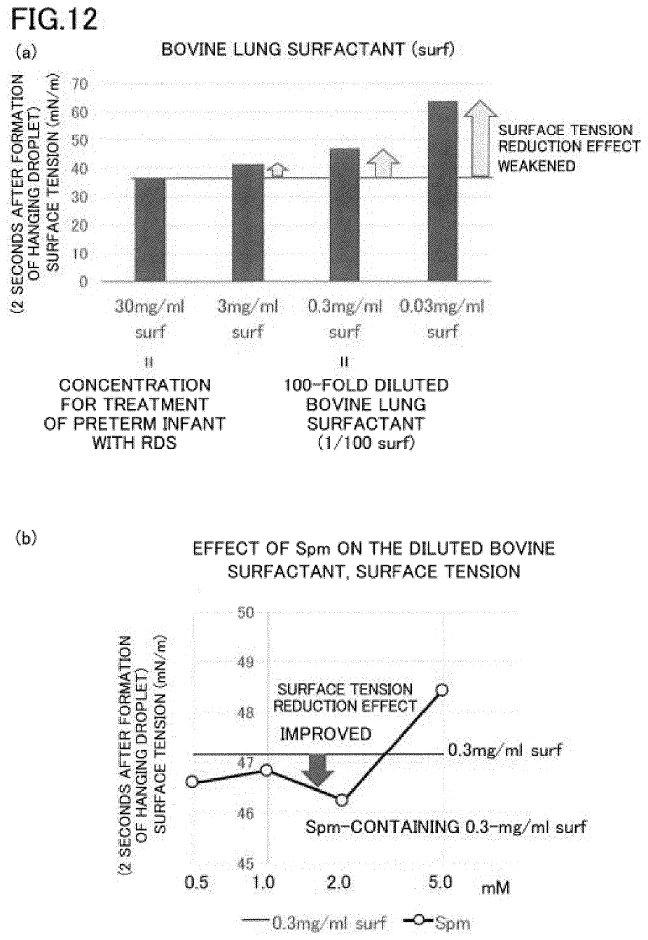

In FIG. 12a, it was demonstrated that the surface tension reduction effect of bovine lung surfactant decreases due to dilution. The surface tension reduction effect (the leftmost column) of the 30-mg/ml bovine lung surfactant solution (surf), which is used in treatment of a preterm infant with RDS, became weakened depending on the degree of dilution (arrows with an upward arrowhead in FIG. 12a). In FIG. 12b, influence of Spm on the surface tension of the 0.3 mg/ml surf, which mimics in vitro ARDS model, was studied. The presence of 0.5, 1, and 2 mM of Spm reduced surface tension (FIG. 12b).

Spm alone does not have such a surface tension reduction effect and was also demonstrated in the experiment (e.g., in the case of 0.5 mM of Spm, 64 mN/m, 0-10000 msec). It is assumed that 5 mM of Spm administered to a lung in ARDS model was mixed with physiological saline remaining in pulmonary alveolar cavities, so that the concentration of Spm became several millimoles lower than 5 mM, and that Spm exhibited the surface tension reduction defected by cooperated with the twice diluted endogenous pulmonary surfactant remaining in the lung (FIG. 12). It is considered that whereby the collapse of pulmonary alveoli was ameliorated (FIG. 9d), and lung compliance was improved (open columns in FIG. 10).

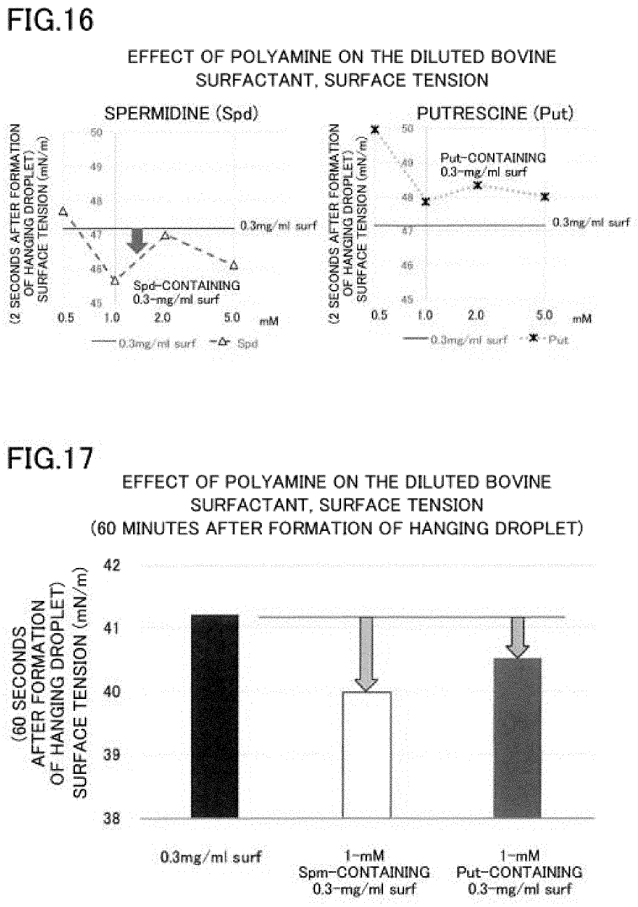

FIG. 16 shows influence of Spd and Put on surface tension in a diluted surfactant environment in which the surface tension reduction effect is weakened. It was demonstrated that Spd also has the surface tension reduction effect (left in FIG. 16).

FIG. 17 shows influence of Spm and Put on surface tension in a diluted surfactant environment in which the surface tension reduction effect is weakened. The surface tension reduction effect of Put was observed on several tens of seconds after formation of a hanging droplet (right column in FIG. 17).

Like Spm, Spd or Put alone does not have the surface tension reduction effect. It is considered that the surface tension reduction effect found in the above experiments (arrow with a downward arrowhead in the left graph of FIG. 16, and arrows with a downward arrowhead in the right columns of FIG. 17) is attributed to cooperation with diluted bovine lung extract surfactant.

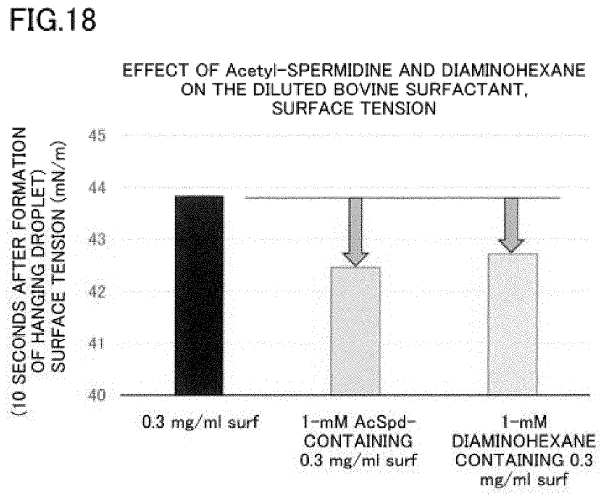

FIG. 18 shows a study of influence of a polyamine that is not present in the higher animals and an acetylated polyamine, on surface tension in a diluted surfactant environment in which the surface tension reduction effect is weakened. N1-acetylspermidine (N1-AcSpd), which is a representative example of an acetylated polyamine, and diaminohexane, which is a representative example of a polyamine that is not present in the higher animals, were used. FIG. 18 shows surface tension as measured 10 seconds after production of a hanging droplet. The left column shows the surface tension of a 0.3 mg/ml bovine lung surfactant (0.3 mg/ml Surf). The presence of 1 mM of N1-AcSpd in a 0.3 mg/ml bovine lung surfactant led to a reduction in surface tension like Spm (center column in FIG. 18). Likewise, the presence of 1 mM of diaminohexane in a 0.3 mg/ml bovine lung surfactant led to a reduction in surface tension (right column in FIG. 18).

One millimole of N1-AcSpd or 1 mM of diaminohexane alone does not have the surface tension reduction effect (63 mN/m, 62 mN/m, 0-10000 msec). The surface tension reduction effect found in this experiment (arrow with a downward arrowhead in FIG. 18) is considered to be attributed to cooperation with diluted bovine lung surfactant. Thus, it was demonstrated that a polyamine that is not present in living organisms and an acetylated polyamine can cooperate with pulmonary surfactant to exhibit the effect of reducing surface tension.

Thus, it was demonstrated that Spm, which is a representative polyamine that is endogenous to the pulmonary alveolar cavities, has the effect of recovering surface activity that was lost due to dilution of pulmonary surfactant (FIG. 12b). It is inferred that this surface mechanical recovery in vivo leads to an improvement in lung compliance (FIGS. 5, 6, and 10), resulting in prevention of the collapse of the lungs (FIG. 9) and amelioration of gas exchange dysfunction (hypoxemia) (FIGS. 7 and 8).

Spd and Put, which are endogenous to the pulmonary alveolar cavities, also have the effect of recovering surface activity that was lost due to dilution of pulmonary surfactant (FIGS. 16 and 17), and therefore, are expected to improve lung compliance, prevent the collapse of the lungs, and ameliorate gas-exchange.

In addition to major polyamines present in the higher animals (claim 3), acetylated polyamines (claim 4) and polyamines that are not present in the higher animals (claim 5), have the effect of recovering surface activity that was lost due to dilution of pulmonary surfactant (FIG. 18), and therefore, are expected to improve lung compliance, prevent collapse of the lungs, and ameliorate hypoxemia.

In addition, no polyamine alone has the surface tension reduction effect. Therefore, it is inferred that polyamines and surfactant that are endogenous to the pulmonary alveolar cavities cooperate together to contribute to a reduction in surface tension. It has been reported that replacement therapy using a bovine lung extract artificial surfactant or totally-synthesized artificial surfactant is not effective to patients with ARDS. In the above examples, administration of several millimoles of a polyamine to pulmonary alveolar cavities alone led to an improvement in lung compliance, prevention of the collapse of the lungs, and amelioration of gas exchange dysfunction. Therefore, the above results show that the presence of several millimoles of a polyamine in pulmonary alveolar cavities is crucial irrespective of somewhat dilution of surfactant. It is known that the polyamine concentration is high in individuals during developmental processes. While the concentration of polyamines endogenous to the pulmonary alveolar cavities is possibly sufficient in IRDS, the polyamine concentration in the pulmonary alveolar cavities is possibly low due to exudates in ARDS, which it is considered makes the artificial pulmonary surfactant replacement therapy effective and ineffective, respectively.

The result of the in vitro experiment (Example 8) demonstrated that polyamines cooperate with pulmonary surfactant to exhibit the surface tension reduction effect. When active pulmonary surfactant is diluted, transairway administration of, for example, Spm alone at an appropriate concentration leads to an improvement in lung compliance and oxygenation (FIGS. 5, 7, and 9), i.e., the effect of the pharmaceutical agent containing a polyamine according to the present invention is expected. It is inferred that the presence of pulmonary surfactant that is inactive due to adhesion of fibrin or the like weakens the effect of polyamine administration. In such a case, it is inferred that addition of a phospholipid or a phospholipid component, or an exogenous surfactant used in conventional surfactant replacement therapies, to the pharmaceutical agent containing a polyamine according to the present invention is effective in treatment of ARDS. In addition, when a pharmaceutical agent containing a polyamine to which a bovine lung extract surfactant containing a phospholipid is added was administered to models with normal ARDS via the transairway route, the effect of improving lung compliance (FIG. 6) and the effect of improving oxygenation (FIG. 8) were observed.

### (9) Example 9

In Example 9, polyamines were analyzed in terms of the surface tension reduction effect (in vitro), based on the polyamine composition ratio in pulmonary alveoli of a rat under general anesthesia, and the lung compliance improvement effect of optimized administration of polyamines to pulmonary alveoli was studied.

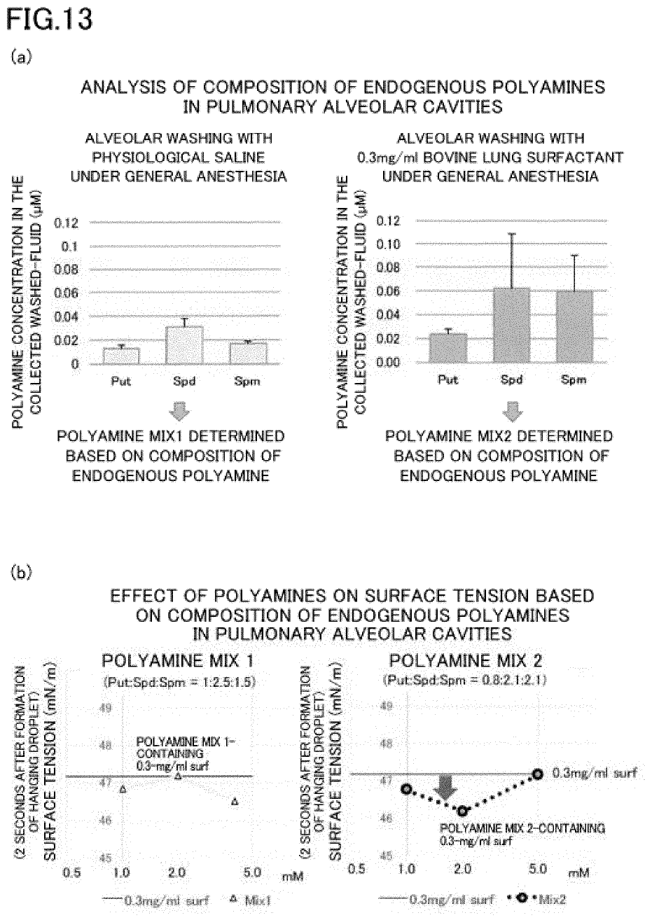

A tracheal cannula was inserted into a rat under general anesthesia. When the respiratory and circulatory dynamics of the rat were stable under artificial ventilation, 20 ml/kg of a pulmonary alveolar washing solution (physiological saline) was injected/suctioned consecutively 5 times within 2 minutes through the tracheal cannula. The polyamine composition in the collected washed-fluid was analyzed (left in FIG. 13a). Based on this, a polyamine composition ratio mix1 was determined to be Put:Spd:Spm=1:2.5:1.5 to a similar extent as endogenous one when pulmonary alveolar washing was conducted with physiological saline.

A tracheal cannula was inserted into a rat under general anesthesia. When the respiratory and circulatory dynamics of the rat were stable under artificial ventilation, 20 ml/kg of a pulmonary alveolar washing solution (100-fold diluted bovine lung extract surfactant) was injected/suctioned consecutively 5 times within 2 minutes through the tracheal cannula. The polyamine composition in the collected washed-fluid was analyzed (right in FIG. 13a). Based on this, a polyamine composition ratio mix2 was determined to be Put:Spd:Spm=0.8:2.1:2.1, to a similar extent as endogenous one when pulmonary alveolar washing was conducted with the 100-fold diluted bovine lung extract surfactant.

For the polyamine compositions mix1 and mix2, surface tension was analyzed using the method described in Example 8 in order to obtain an optimum concentration having a high surface tension reduction effect.

The following polyamine mix-containing 100-fold diluted bovine lung extract artificial surfactants were used.

A polyamine mix 1-containing 100-fold diluted bovine lung extract artificial surfactant is indicated, in the drawings and specification, by “polyamine mix 1 (Put:Spd:Spm=1:2.5:1.5)-containing 0.3-mg/ml pulmonary surfactant” or “polyamine mix 1-containing 0.3 mg/ml surf.”

A polyamine mix 2-containing 100-fold diluted bovine lung extract artificial surfactant is indicated, in the drawings and specification, by “polyamine mix 2 (Put:Spd:Spm=0.8:2.1:2.1)-containing 0.3-mg/ml pulmonary surfactant” or “polyamine mix 2-containing 0.3 mg/ml surf.”

An in vivo experiment below was conducted in order to determine whether or not the polyamine composition mix2, which has an optimum surface tension reduction effect obtained in the above in vitro experiment, has the lung compliance improvement effect.

A tracheal cannula was inserted into a rat under general anesthesia. When the respiratory and circulatory dynamics of the rat were stable under artificial ventilation, 20 ml/kg of a pulmonary alveolar washing solution (physiological saline containing an optimum concentration of the polyamine mix 2) was injected/suctioned consecutively 5 times within 2 minutes through the tracheal cannula. After the washing solution remaining in the trachea was suctioned out, artificial ventilation was immediately resumed. Thus, an ARDS model whose pulmonary alveoli were washed with the polyamine mix 2 having the optimum concentration was produced.

The above ventilation was conducted by inverse ratio ventilation without using PEEP. The ventilation was conducted using ambient air (inhaled oxygen concentration FiO₂: 0.21) without oxygen administration. The intra-airway pressure was measured over time. Influence of the polyamines was analyzed, where dynamic lung compliance was used as a measure of the distensibility of the lung.

The results are shown in FIGS. 11, 13, 14, and 15.

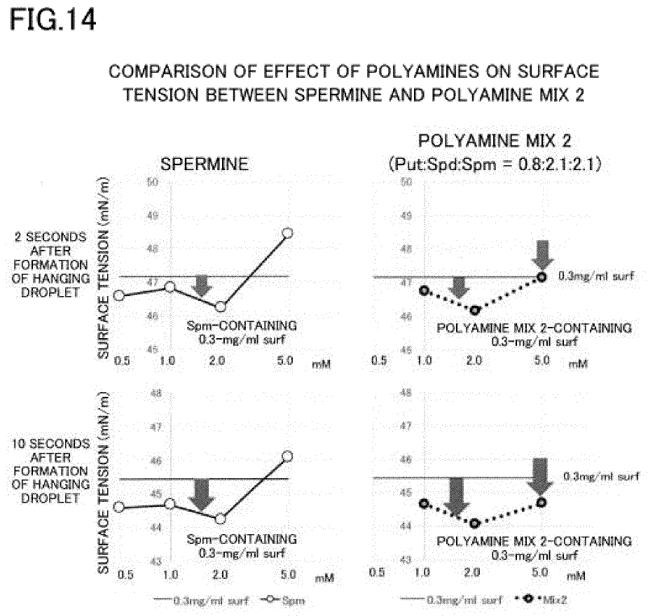

FIG. 11 shows an image of a droplet captured after the lapse of a predetermined period, between the continuous measurement of surface tension using the polyamine mix 2-containing 0.3-mg/ml pulmonary surfactant as described in FIGS. 13 and 14 below. A droplet (right in FIG. 11) of the 2-mM polyamine mix 2-containing 0.3-mg/ml pulmonary surfactant extended further in the gravitational direction than that of the 0.3-mg/ml pulmonary surfactant (left in FIG. 11).

FIG. 13a shows the types and concentrations of endogenous polyamines in the collected washed-fluids when pulmonary alveolar washing was conducted using physiological saline (left in FIG. 13a) or physiological saline containing 0.3-mg/ml pulmonary surfactant (right in FIG. 13a). Although washing was conducted under the same conditions (20 ml/kg, 5 consecutive times within 2 minutes), the polyamine concentration was higher when washing was conducted using the bovine pulmonary surfactant-containing physiological saline (right in FIG. 13a). It is inferred that polyamines endogenous to the pulmonary alveolar cavities have affinity to pulmonary surfactant. Comparison of the compositions shows that a greater amount of endogenous Spm was detected when washing was conducted using the bovine pulmonary surfactant-containing physiological saline (right FIG. 13a), which suggests the possibility that Spm has high affinity to pulmonary surfactant. The composition ratio mix1 (Put:Spd:Spm=1:2.5:1.5) and the composition ratio mix2 (Put:Spd:Spm=0.8:2.1:2.1) were determined based on the compositions of endogenous polyamines in the respective collected washed-fluids (FIG. 13a).

FIG. 13b shows influence of the polyamine mix 1 and mix 2 on surface tension in a diluted surfactant environment in which the surface tension reduction effect is weakened. A 0.3-mg/ml bovine pulmonary surfactant containing the polyamine composition mix1 or mix2 was used to analyze influence on surface tension. The composition mix2 exhibited the surface tension reduction effect continuously over a wide concentration range, compared to the composition mix1 (right in FIG. 13b).

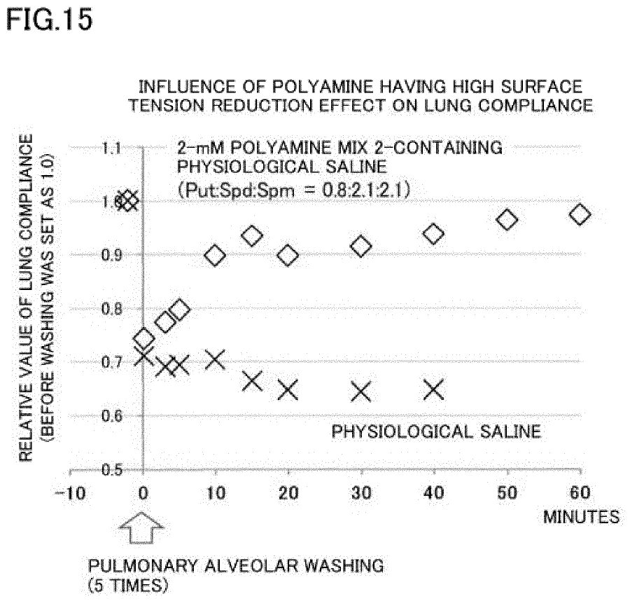

FIG. 14 shows comparison of influence of Spm and the polyamine mix 2 on surface tension in a diluted surfactant environment in which the surface tension reduction effect is weakened. The polyamine composition mix2 exhibited the surface tension reduction effect continuously over a wide concentration range, compared to Spm (the upper and lower figures on the right side of FIG. 14). Using the 2-mM polyamine mix 2 (Put:Spd:Spm=0.8:2.1:2.1), an in vivo effect was analyzed (FIG. 15).

FIG. 15 shows influence in vivo, of the 2-mM polyamine mix 2 (Put:Spd:Spm=0.8:2.1:2.1) having the highest surface tension reduction effect, on lung compliance. As shown in FIG. 15, lung compliance decreased immediately after washing due to a pulmonary alveolar washing operation (open diamonds and cross in FIG. 15). On washing with a 2-mM polyamine mix 2 (Put:Spd:Spm=0.8:2.1:2.1)-containing physiological saline, lung compliance subsequently recovered (open diamonds in FIG. 15). Furthermore, when lung compliance before washing was set as 1.0, the time it took to recover to 0.9 after washing with the 2 mM polyamine mix 2 was as short as about 10 minutes.

### INDUSTRIAL APPLICABILITY

The embodiments are useful in treatment of acute respiratory distress syndrome (ARDS), lung diseases caused by dysfunctional endogenous pulmonary alveolar surfactant, multiple organ dysfunction syndrome (MODS), and cardiogenic pulmonary edema. The embodiments are also useful in prophylaxis of a decrease in lung compliance after a bronchoalveolar lavage (BAL) test.

### DESCRIPTION OF REFERENCE CHARACTERS

1 MEDICAL SOLUTION
2 MEDICAL SOLUTION RESERVOIR
3 AIR INTRODUCTION TUBE
4 AIR DISCHARGE TUBE
7 AIR INTRODUCTION OPENING
8 SPRAY OPENING
9 AIR FLOW PASSAGE
12 TIP PORTION
13 OCULAR PORTION
14 OPERATION PORTION
15 FEED OPENING
16 INSERTION TUBE PORTION

1. A pharmaceutical agent for prophylaxis and/or treatment of a pathological condition with decreased lung compliance, comprising a polyamine.

2. The pharmaceutical agent of claim 1, wherein
the pathological condition with decreased lung compliance is acute respiratory distress syndrome (ARDS).

3. The pharmaceutical agent of claim 1, wherein
the polyamine includes at least one of spermine (Spm), spermidine (Spd), and putrescine (Put).

4. The pharmaceutical agent of claim 1, wherein
the polyamine includes at least one of acetylputrescine, N1-acetylspermidine, N8-acetylspermidine, and N1-acetyl spermine.

5. The pharmaceutical agent of claim 1, wherein
the polyamine includes at least one of 1,3-diaminopropane, diaminohexane, cadaverine, agmatine, caldine, homospermidine, aminopropylcadaverine, thermine, thermospermine, canavalmine, aminopentylnorspermidine, N,N-bis(aminopropyl)cadaverine, homospermine, caldopentamine, homocaldopentamine, caldohexamine, and homocaldohexamine.

6. The pharmaceutical agent of claim 1, further comprising a phospholipid.

7. The pharmaceutical agent of claim 6, wherein
the phospholipid includes at least one of dipalmitoyl-phosphatidylcholine (DPPC), phosphatidylcholines (PC), glycerol (PG), distearoylphosphatidylcholine (DSPC), distearoylphosphatidylglycerol (DSPG), dimyristoyl-phosphatidylethanolamine (DMPE), dipalmitoylphosphatidylethanolamine (DPPE), dioleylphosphatidylethanolamine (DOPE), distearoylphosphatidylethanolamine (DSPE), diarachidoylphosphatidylethanolamine (DAPE) or dilinoleylphosphatidylethanolamine (DLPE), dilauroyl-phosphatidylcholine (DLPC), dimyristoyl-phosphatidylcholine (DMPC), diarachidoyl-phosphatidylcholine (DAPC), dioleyl-phosphatidylcholine (DOPC), dimyristoylphosphatidylserine (DMPS), diarachidoylphosphatidylserine (DAPS), dipalmitoylphosphatidylserine (DPPS), distearoylphosphatidylserine (DSPS), dioleylphosphatidylserine (DOPS), dipalmitoylphosphatidic acid (DPPA), dimyristoylphosphatidic acid (DMPA), distearoylphosphatidic acid (DSPA), diarachidoylphosphatidic acid (DAPA), dimyristoylphosphatidylglycerol (DMPG), dipalmitoylphosphatidylglycerol (DPPG), dioleyl-phosphatidylglycerol (DOPG), dilauroylphosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), and dioleylphosphatidylinositol (DOPI).

8. A medical solution for washing pulmonary alveoli, comprising a polyamine.

9. The medical solution of claim 8, wherein
the polyamine includes at least one of spermine (Spm), spermidine (Spd), and putrescine (Put).

10. A nebulizer comprising:
an air flow passage extending from an air introduction opening to a spray opening; and
a medical solution reservoir configured to contain a medical solution and mist the medical solution, wherein
air introduced from the air introduction opening is mixed with the medical solution misted in the medical solution reservoir, and the mixed air is discharged from the spray opening, and
the medical solution has a polyamine.

11. The nebulizer of claim 10, wherein
the polyamine includes at least one of spermine (Spm), spermidine (Spd), and putrescine (Put).

12. A bronchoscope comprising:
a tube-shaped insertion tube portion configured to be inserted into the trachea and then a bronchus, through the mouth or nose and then the throat, or through a tracheal cannula;
a spray hole provided in the insertion tube portion and penetrating from a rear end to a front end of the tube, and configured to transfer a medical solution from the rear end to the front end;
a spray opening provided at a tip portion of the insertion tube portion and configured to discharge the medical solution in the form of a mist into the bronchus; and
a feed opening through which the medical solution is fed into the spray hole of the insertion tube portion,
wherein
the medical solution has a polyamine.

13. The bronchoscope of claim 12, wherein
the polyamine includes at least one of spermine (Spm), spermidine (Spd), and putrescine (Put).

14. The pharmaceutical agent of claim 2, wherein
the polyamine includes at least one of spermine (Spm), spermidine (Spd), and putrescine (Put).

15. The pharmaceutical agent of claim 2, wherein
the polyamine includes at least one of acetylputrescine, N1-acetylspermidine, N8-acetylspermidine, and N1-acetyl spermine.

16. The pharmaceutical agent of claim 2, wherein
the polyamine includes at least one of 1,3-diaminopropane, diaminohexane, cadaverine, agmatine, caldine, homospermidine, aminopropylcadaverine, thermine, thermospermine, canavalmine, aminopentylnorspermidine, N,N-bis(aminopropyl)cadaverine, homospermine, caldopentamine, homocaldopentamine, caldohexamine, and homocaldohexamine.

17. The pharmaceutical agent of claim 2, further comprising a phospholipid.

18. The pharmaceutical agent of claim 3, further comprising a phospholipid.

19. The pharmaceutical agent of claim 4, further comprising a phospholipid.

20. The pharmaceutical agent of claim 5, further comprising a phospholipid.
